(19) United States
(12) Patent Application Publication
(10) Pub. No.: US 2026/0087753 A1
(43) Pub. Date: Mar. 26, 2026

(54) SYSTEMS AND METHODS OF ALIGNING A PATIENT WITH A MEDICAL DEVICE LEVERAGING OUTSIDE-IN TRACKING

(71) Applicant: University of Florida Research Foundation, Incorporated, Gainesville, FL (US)

(72) Inventor: Perry B. Johnson, Gainesville, FL (US)

(73) Assignee: University of Florida Research Foundation, Incorporated, Gainesville, FL (US)

(21) Appl. No.: 19/113,540

(22) PCT Filed: Sep. 19, 2023
(86) PCT No.: PCT/US2023/033103

§ 371 Date: Mar. 20, 2025

Related U.S. Application Data
(60) Provisional application No. 63/408,711, filed on Sep. 21, 2022.

(51) Int. Cl. G06T 19/00; A61N 5/10

(52) U.S. Cl. CPC G06T 19/006; A61N 5/1039; G06T 2210/41

(57) ABSTRACT

According to at least one aspect, a method is provided for registering a patient with a medical device for treating the patient. The method includes accessing pose information, wherein the pose information is of a patient, a mixed, reality display, or both, and. wherein the pose information is generated by a. tracking system external to the mixed reality display, registering a pose of a 3D representation of the patient with the medical device using, at least in part, the pose information, and generating, based on results of the registering, a mixed reality visualization of the 3D representation of the patient and the device.

### CROSS-REFERENCE TO RELATED APPLICATIONS

This application claims priority to U.S. Provisional Application No. 63/408,711, filed Sep. 21, 2022, which is hereby incorporated herein by reference in its entirety.

### BACKGROUND

It is often necessary to register a patient with a medical device for medical observation or to perform a medical procedure. For example, linear accelerators deliver radiation treatments to patients using radiation beams. In order to deliver the radiation beams to a desired part of the patient's anatomy (e.g., to a portion containing a tumor), the patient needs to be aligned to the linear accelerator and in a manner that reproduces a specific posture. As another example, a computed tomography (CT) simulator can use x-ray imaging to create a representation of a patient and tumor. In certain applications, the patient must be aligned to the CT simulator in a manner that also reproduces a specific posture.

### SUMMARY

According to at least one aspect, an apparatus and/or a method is provided for registering a patient with a medical device for treating the patient. Pose information is accessed, wherein the pose information is of a patient, a mixed reality display, or both, and wherein the pose information is generated by a tracking system external to the mixed reality display. A pose of a 3D representation of the patient is registered with the medical device using, at least in part, the pose information. Based on results of the registering, a mixed reality visualization of the 3D representation of the patient and the medical device is generated.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

Various aspects and embodiments will be described with reference to the following figures. It should be appreciated that the figures are not necessarily drawn to scale. Items appearing in multiple figures are indicated by the same or a similar reference number in all the figures in which they appear.

FIG. 1 is a diagram of an exemplary system for registering a 3D representation of a patient with a medical device, according to some embodiments of the techniques described herein.

FIG. 2 shows exemplary tracking devices, according to some embodiments of the techniques described herein.

FIG. 3 is a diagram of an exemplary medical device with a tracking device mounted to the medical device, according to some embodiments of the techniques described herein.

FIG. 4 is a flow chart of an exemplary computerized method for registering a 3D representation of a patient with a medical device, according to some embodiments of the techniques described herein.

FIG. 5 is a diagram showing an exemplary real-world coordinate space for the physical space of the medical device and a machine coordinate space for the head mounted display, according to some embodiments of the techniques described herein.

FIG. 6A is an image showing a 3D representation of an anthropomorphic phantom from a first viewpoint in a physical scene, according to some embodiments of the techniques described herein.

FIG. 6B is an image showing the 3D representation of an anthropomorphic phantom of FIG. 6A after adjustment and from a second viewpoint in the physical scene, according to some embodiments of the techniques described herein.

FIG. 7 is a flow chart showing a computerized method for registering the 3D representation and tracking the registration of the 3D representation over time, according to some embodiments of the techniques described herein.

FIG. 8 includes images of an exemplary proton therapy device that can be used with the 3D representation registration techniques described herein, according to some embodiments.

FIG. 9 shows an illustrative implementation of a computer system that may be used in connection with any of the embodiments of the techniques provided herein.

FIG. 10 is a flow chart of an exemplary computerized method for providing alignment information to aid with aligning a patient with a 3D representation of the patient, according to some embodiments of the techniques described herein.

FIG. 11A is an image showing a visual indication of a portion of a patient that is aligned to a 3D representation of the patient within an acceptable alignment threshold, according to some embodiments of the techniques described herein.

FIG. 11B is an image showing visual indications of both acceptable an unacceptable portions of a patient being aligned to a 3D representation, according to some embodiments of the techniques described herein.

FIG. 12 is a diagram of an exemplary system for registering a 3D representation of a patient with a medical device using an external tracking system, according to some embodiments.

FIG. 13 is a flow chart of an exemplary computerized method for generating a mixed reality visualization of a 3D representation of the patient and the medical device using outside-in tracking, according to some embodiments.

FIG. 14A is an image showing an exemplary real-world scene that includes an anthropomorphic phantom aligned with a medical device, according to some embodiments.

FIG. 14B is an image showing a mixed reality rendering that includes a 3D representation of the anthropomorphic phantom and a 3D representation of a marker, according to some embodiments.

FIG. 15 is a flow chart of an exemplary computerized method for confirming registration of 3D representations with the medical device during mixed reality rendering, according to some embodiments.

### DETAILED DESCRIPTION

The inventors have appreciated challenges with aligning patients with medical devices, such as linear accelerators, CT simulators, and/or the like. In particular, patient alignment can be challenging due to the non-rigid alignment of patient anatomy. A conventional approach that can be used for patient alignment is surface-guided radiation therapy (SGRT). SGRT generally uses a ceiling-mounted illumination system that emits known light patterns, and uses the captured reflections to reconstruct anterior portions of the patient's surface through stereophotogrammetry. The reconstructed surface can then be matched to a reference surface (e.g., determined based on the patient's outer body contour during a simulation CT scan), and displayed on a 2D monitor to help guide alignment of the patient with the device.

The inventors have discovered and appreciated various deficiencies with such conventional techniques. In particular, since the illumination system is mounted to the ceiling and therefore cannot move in the environment, SGRT approaches can suffer from field of view limitations. Relatedly, SGRT approaches can suffer from camera obstructions, such that objects between the illumination sources and the patient can create shadows that can negatively impact the reconstructed surface. SGRT approaches also typically cannot track posterior surfaces of the patient, which therefore limits alignment to only using anterior surfaces (e.g., which reduces the accuracy with which a patient can be aligned using SGRT), SGRT approaches also require the clinician to redirect their gaze from the patient to the 2D monitor displaying the reconstructed surface and the reference surface, which can make alignment cumbersome and time consuming. Further, SGRT approaches can also be expensive, costing upwards of $100K, if not more.

The inventors have appreciated that mixed reality (MixR) applications can be used to improve patient alignment. MixR applications can generally provide for visually overlaying three-dimensional (3D) representations (e.g., virtual objects, such as holograms) on a physical environment. Various displays, such as optical-see-through or video-pass-through head mounted displays can be used for MixR applications. The head mounted displays often use a variety of sensors to map the surroundings, track physical objects, and render 3D representations at specific locations. MixR applications can enable such attributes while a user dynamically navigates a physical scene, thus affording natural viewing and interaction with 3D representations in the physical scene.

MixR can be used to improve situational awareness and/or information management by providing visual content in the user's viewing space. In surgical applications, for example, virtual two-dimensional (2D) panels can be used to display pre-operative planning materials (images, notes, etc.) directly within the surgeon's field of view. Displaying such virtual 2D panels can avoid the surgeon needing to redirect their gaze from the surgical site (e.g., to view surgical notes, etc.) during surgery. As another example, MixR techniques can be used for surgical navigation to track the orientation and/or positioning of external tools in relationship to a patient. In such applications, a 3D representation can be registered to the patient through inside-out tracking (e.g., using the sensors available on the head mounted display). Inside-out tracking differs from an outside-in approach in which external hardware beyond that of the head mounted display is required to track and register objects. Outside-in navigation systems can suffer from various deficiencies, such as increasing clutter in the operational space, suffering line-of-sight obstructions issues, precluding optimal ergonomics, lacking portability, and/or can be prohibitively expensive.

Types of inside-out tracking used for MixR include marker-based tracking and marker-free tracking. With marker-based tracking, the position of a 3D representation can be determined via its relationship to a known object (e.g., to a known tracking object) placed in the physical space and subsequently recognized through feature detection. For marker-free tracking, the system can track the position of a 3D representation based on aspects of the physical scene. Marker-free tracking often needs to address a chicken-or-egg problem whereby a map of the scene is needed for localization but a pose estimate of the head mounted display (in relation to a map) is needed for mapping. This problem can be resolved by estimating the spatial relationships between the head mounted display and multiple key points identified within a series of viewpoints. Increasing either the number of viewpoints or the availability/quality of features within an environment can improve this estimate. Conventional head mounted displays can include various sensors for maker-free tracking, such as IR cameras, RGB cameras, inertial sensors, and/or the like.

The inventors have developed MixR applications, which can leverage marker-based and/or marker-free tracking, that can be used to align a patient to a medical device. Accordingly, the inventors have conceived and developed new technology to align patients with medical devices that improves upon the various problems and deficiencies with conventional alignment approaches. The techniques described herein approach the alignment problem from the opposite direction than that used by conventional approaches in many medical applications. In particular, the techniques described herein align the patient to the device, rather than trying to align the device to the patient. In particular, because patient anatomy is not always rigid, device-to-patient alignment is limited in scope, and is typically confined to rigid anatomy (e.g., the skull) or highly localized areas. Conversely, in patient-to-device alignment using the techniques described herein, the patient can be manipulated in a non-rigid manner to achieve the correct posture needed for further global rigid registration.

In some embodiments, the techniques described herein provide for registering a patient with a medical device using a 3D representation (e.g., a hologram) of the patient. The 3D representation can be generated based upon the external body contour of a patient, such as by deriving the 3D representation from a patient's planning CT dataset. The hologram can be registered to a medical device and viewed directly through a head mounted display to provide a reference posture to which the patient can be matched and aligned during treatment setup. Obstructions in the scene, potential field of view limitations, and/or ergonomic limitations can be addressed through use of the head mounted display, which can leverage inside-out-tracking to allow the user to view the patient and 3D representation directly from multiple angles without needing to take their gaze off the patient during alignment.

To register the origin of the 3D representation with the medical device, in some embodiments an initialization procedure can be performed that uses marker-based tracking. For example, a feature detection algorithm can be used that leverages an RGB camera on the head mounted display to detect and track known objects (e.g., tracking devices) placed in a physical space. For example, tracking devices such as a 3D object with QR-codes can be attached to the medical device such that the tracking device has a known offset to a point of interest of the medical device (e.g., to the radiation isocenter of a linear accelerator). By viewing the tracking device with the head mounted display, the 3D representation can be automatically registered and rendered at the appropriate location in the physical scene and used to easily and naturally align the patient. In some embodiments, the patient isocenter can be linked to the 3D representation and registered to the radiation isocenter, which in-turn allows the patient isocenter to be linked to the radiation isocenter.

The inventors have further developed techniques for quantifying the difference between a registered 3D representation of the patient and a real-time 3D representation of the patient (e.g., the surface contour of the patient), where the real-time 3D representation that is generated during patient alignment to aid with alignment. In some embodiments, the quantified differences (e.g., distances between associated points of the real-time and registered 3D representations) are visually displayed as part of a MixR rendering to aid with alignment. For example, the differences can be visually displayed to a user wearing a head-mounted display. In some embodiments, the visual indicators can indicate a degree of difference between a portion(s) of the patient and the registered 3D representation. For example, the visual indicators can indicate which portion(s) of the patient are properly aligned with the registered 3D representation, as well as which portion(s) of the patient are not properly aligned with the 3D representation (e.g., and therefore require further adjustment). Accordingly, a user can use the visual indicators to adjust the patient in real-time to achieve a sufficient alignment of the patient for medical treatment.

According to some embodiments, the techniques include leveraging information from a tracking system that is external to the mixed reality display (e.g., head mounted display) for use in generating a mixed reality visualization of the 3D representation of the patient with the medical device. For example, pose information from the tracking system, such as pose information of the patient and/or of the mixed reality display, can be used to generate the mixed reality visualization. In some embodiments, marker(s) or tracking sensor(s) can be mounted on and/or associated with the mixed reality display. The tracking system can track the marker(s) in order to track the pose of the mixed reality display. In some embodiments, the frame of reference of the tracking system can be calibrated with the frame of reference of the medical device. The tracking system and/or the medical device can also measure and/or track the patient's surface (e.g., to generate and/or track a real-time 3D representation of the patient). The mixed reality display can receive and/or access pose information of the mixed reality display and/or of the patient and generate a mixed reality visualization of a 3D representation of the patient and the medical device. In some embodiments, the techniques can include calibrating and/or combining the pose estimations of the patient and/or mixed reality display to provide a rendering of the 3D representation of the patient and a real-time 3D representation of the patient's surface.

The inventors have further appreciated that including a 3D representation of a tracking device or marker (e.g., 3D object with QR-codes, infrared-based marker) can allow a user of a head-mounted display to readily ascertain misalignment between the real-world and a mixed reality visualization. Following registration of the 3D representations with the medical device, during marker-free tracking, a misalignment may occur due to drift or an issue with a camera of the head-mounted display providing the mixed reality visualization. The marker has a fixed relationship with the medical device, unlike a patient who may be moving. Thus, any misalignment between the 3D representation of the marker and the real marker in the MixR rendering is visually apparent to the user who can command re-registration (i.e., marker-based realignment), as needed. According to some embodiments, by using an infrared-based marker, marker-free tracking may be eliminated or a duration of marker-free tracking may be reduced, because the user need not look at the marker during adjustment of the patient in order to maintain alignment.

It should be appreciated that the embodiments described herein may be implemented in any of numerous ways. Examples of specific implementations are provided below for illustrative purposes only, It should be appreciated that these embodiments and the features/capabilities provided may be used individually, all together, or in any combination of two or more, as aspects of the technology described herein are not limited in this respect.

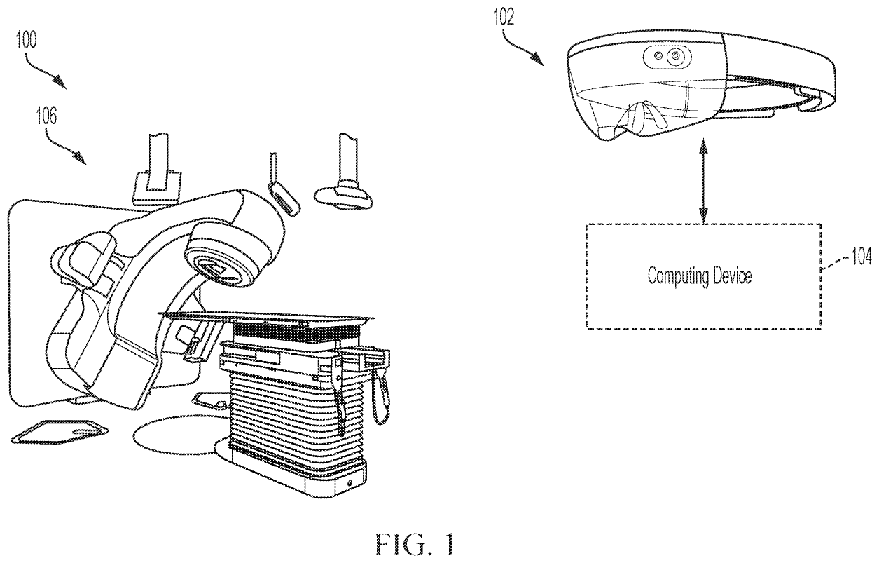

FIG. 1 is a diagram of an exemplary system 100 for registering a 3D representation of a patient with a medical device, according to some embodiments. The system 100 includes a head mounted display 102 with a computing device 104. The computing device 104 is drawn as a dotted box because computing device 104 may be part of the head mounted display 102 and/or a separate computing device that is in communication with the head mounted display 102 (e.g., to implement holographic remoting techniques). Non-limiting examples of head mounted displays 102 that can be used in accordance with the techniques described herein include Microsoft Corporation's HoloLens and HoloLens 2 and Magic Leap Inc.'s Magic Leap One, which all provide for MixR implemented using optical-see-through head mounted displays.

The system 100 also includes a medical device 106 to which the patient is to be aligned. In this example, the medical device 106 is a linear accelerator, but a linear accelerator is used for illustrative purposes only and is not intended to be limiting, as the techniques can be used with any type of medical device. In particular, it should be appreciated that the techniques described herein can be used for any type of treatment where a patient needs to be disposed in a specific patient posture. For example, the techniques can be used with other medical devices such as CT simulators, proton therapy systems, magnetic resonance imaging (MRI) simulators, other types of radiation therapy devices, and/or the like.

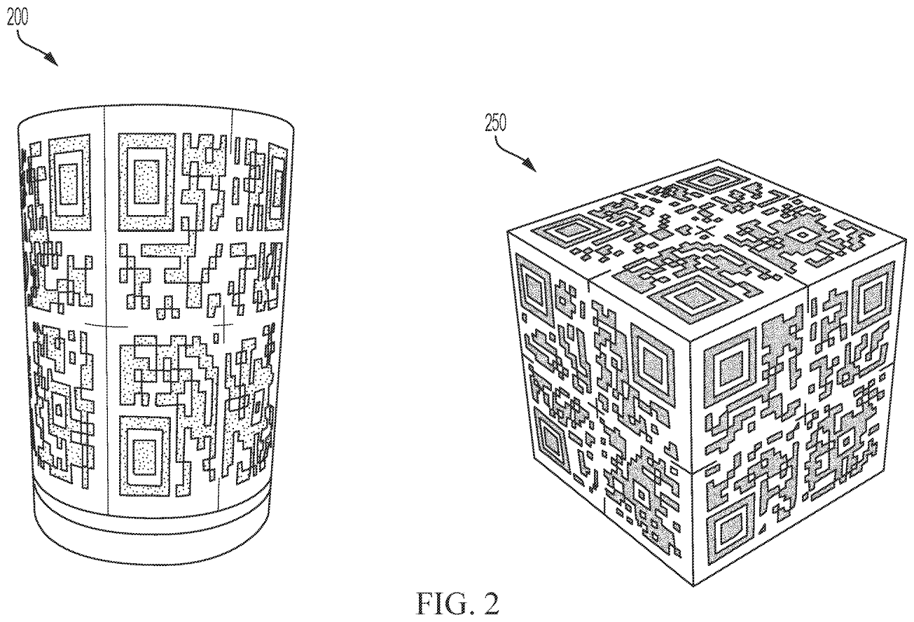

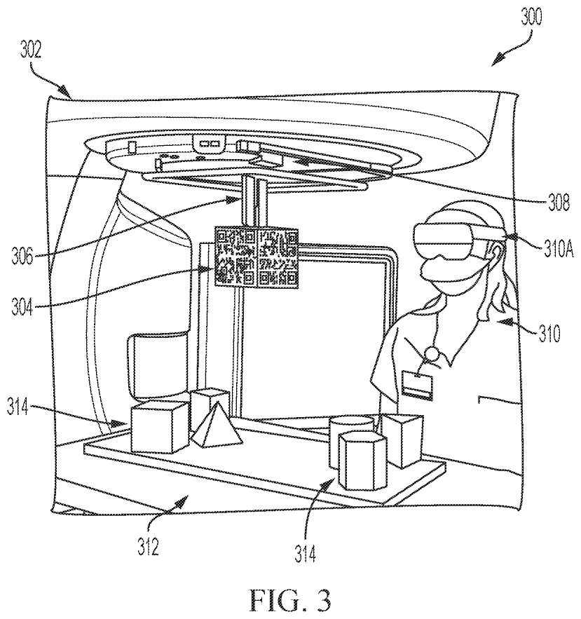

In some embodiments, tracking devices can be used to facilitate registration of a 3D representation of a patient to the medical device. FIG. 2 shows exemplary tracking devices 200 and 250, according to some embodiments. As shown in these examples, tracking devices 200 and 250 each include a set of two-dimensional barcodes. The tracking devices can be of various shapes and sizes, as illustrated in these examples with tracking device 200 having a cylindrical shape and tracking device 250 having a cubic shape. While FIG. 2 shows examples of 3D tracking devices with barcodes, it should be appreciated that this is for exemplary purposes only, and various other types of tracking devices can be used, including image-based optical trackers, shape-based optical trackers, radio frequency or infra-red (IR) trackers, and/or the like. Therefore, the techniques can be used with image-based tracking techniques, IR-based tracking techniques, and/or the like According to some embodiments, one or more tracking devices can be mounted to the medical device. In some embodiments, the tracking device can be mounted using an accessory mounting portion of the device. FIG. 3 is a diagram 300 of an exemplary medical device 302 with a tracking device 304 mounted to the medical device 302, according to some embodiments. An arm 306 mounts the tracking device 304 to an accessory mounting portion 308 of the tracking device to rigidly mount the tracking device 304 to the medical device 302. It should be appreciated that the mounting configuration shown in FIG. 3 is for exemplary purposes only and is not intended to be limiting. The tracking device 304 can be mounted to the medical device 302 using any desired technique. For example, in some embodiments, other components can be used to mount the tracking device 304 to the medical device 302 as necessary (e.g., screws, bolts, brackets, hinges, articulating arms, stands, etc.). In some embodiments, the tracking device 304 can be mounted to different locations of the medical device 302 other than that shown in FIG. 3 (e.g., to the bed of the medical device 302 to a mounting arm, to a side of the medical device 302, etc.). Further, in some embodiments, the tracking device 304 can be disposed near, but not mounted to, the medical device 302. For example, the tracking device 304 can be mounted on a free-standing stand, placed on an object in the scene, and/or the like.

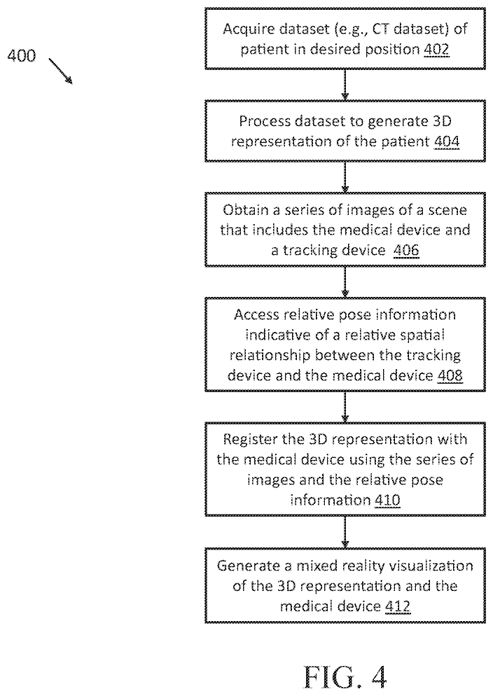

In some embodiments, the patient alignment techniques include generating a 3D representation of the patient anatomy and registering the 3D representation to the medical device so that the patient can be aligned to the medical device using the 3D representation. FIG. 4 is a flow chart of an exemplary computerized method for registering a 3D representation of a patient with a medical device, according to some embodiments. At step 402, a computing device (e.g., the computing device 104 in FIG. 1) acquires a dataset of a patient in a desired position. The dataset can be, for example, a CT dataset of the patient.

At step 404, the computing device processes the dataset to generate a 3D representation of the patient. In some embodiments, the 3D representation can include 3D coordinates, texture maps, and/or other information about the patient. In some embodiments, the 3D representation can be generated by segmenting a CT dataset to generate a 3D representation of the patient's body contour. The 3D representation can be generated of the patient's body contour since the body contour can be used for medical treatment planning purposes. In some embodiments, the 3D representation can be stored as an electronic file, such as a 3D object (OBJ) file. For example, a CT dataset can be exported as a DICOM structure file and re-formatted as a 3D object. In some embodiments, the number of faces of the 3D representation can be reduced (e.g., for rendering consideration of the head mounted display). In some embodiments, the 3D representation can be loaded onto the head mounted display device for rendering. For example, if the 3D representation is stored as a file (e.g., an OBJ file), then the file can be loaded onto the head mounted display for holographic rendering.

At step 406, the computing device obtains a series of images of a physical scene with which the 3D representation is to be visually displayed to the user. As described herein, the scene typically includes at least portion(s) of the medical device to which a patient is to be aligned, a tracking device, and potentially other objects in the scene. As a result, the series of images include at least the tracking device and likely also include portion(s) of the medical device and/or other components or devices as well. For example, when the person 310 in FIG. 3 views the scene using the head mounted display 310A, the images acquired by imaging device(s) on the head mounted display may capture not only the tracking device 304, but also portions of the medical device 302 near the tracking device 304, such as the arm 306, the accessory mounting portion 308, and/or other portions of the medical device 302 (e.g., a portion of the bed 312 and the objects 314). In some embodiments, the head mounted display includes one or more imaging devices (e.g., RGB cameras) on the head mounted display that are used to capture images of the scene. In some embodiments, the head mounted display can use other techniques to capture information about the tracking devices, such as IR cameras, etc.

At step 408, the computing device accesses relative pose information that includes data of the relative spatial relationship between the pose of the tracking device and the pose of the medical device. The relative spatial relationship between the tracking device and the medical device can indicate (a) a relative pose of the tracking device to the medical device, (b) a relative pose of the medical device to the tracking device, or both. In some embodiments, if the tracking device is mounted to the medical device, a known spatial relationship can be determined (e.g., measured and/or pre-configured) between the tracking device and the medical device. For example, the tracking device can be attached to the medical device (e.g., as depicted in FIG. 3) such that the tracking device has a known offset to a point of interest of the medical device. In some embodiments, the relative spatial relationship includes one or more transformations, mappings, (x, y, z) shifts, and/or the like, between the pose of tracking device and the pose of the medical device. In some embodiments, the relative pose information is provided based on reference points of the devices. For example, the relative pose information can be determined between a center of the tracking device and a treatment location of the medical device, such as a location where radiation is directed by the medical device. For example, the location may be the radiation isocenter of a linear accelerator, which is the point that the gantry rotates around since it can be used to link a treatment plan, patient, and device.

In some embodiments, if the tracking device is not mounted to the medical device, the computing device can determine a spatial relationship between the tracking device and medical device using an alignment technique. For example, the computing device can register the tracking device using a three-point alignment technique. In some embodiments, the three-point alignment techniques include determining the relative pose information based on a laser alignment of the tracking device to the medical device.

At step 410, the computing device registers the 3D representation of the patient with the medical device using the series of images of the scene and the relative pose information between the tracking device and the medical device. In some embodiments, the computing device can run a feature detection algorithm on the images of the scene to detect and track the tracking device in physical space. By viewing the tracking device with the head mounted display, the 3D representation can be automatically registered with the appropriate location in the physical scene.

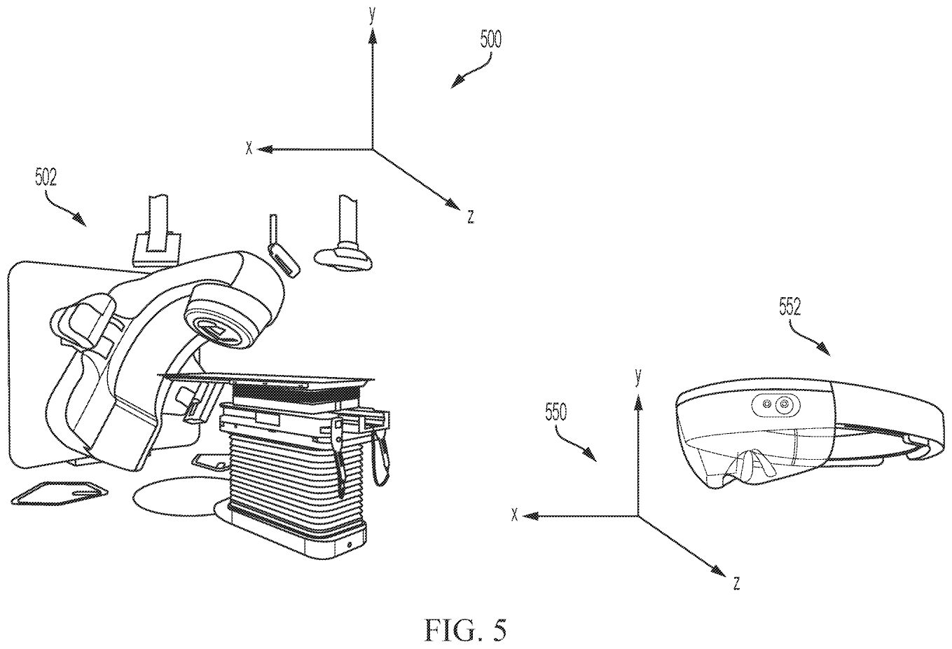

The tracking process can include mapping a pose of the tracking device from physical space to a machine coordinate space of the mixed reality visualization. FIG. 5 is a diagram showing an exemplary real-world coordinate space 500 for the medical device 502 and a machine coordinate space 550 for the head mounted display 552, according to some embodiments. The tracking process can detect and track a pose of the tracking device in the real-world coordinate space 500. The pose of the tracking device in the real-world space can be used to determine a mapping between the 3D representation to the pose of the tracking device in the machine coordinate space 550. For example, the head mounted display can generally use the machine coordinate space 550 to present virtual objects for display on the head mounted display. The computing device can therefore map the pose of the tracking device in the real-world coordinate space 500 to the machine coordinate space 550, and the computing device can register the 3D representation to the pose of the tracking device in the machine coordinate space 550. Such registration process allows the head mounted display to, for example, render MixR experiences with 3D representations (e.g., including holograms and/or other virtual images) in a manner that makes the 3D representations appear as if they are “in” the user's view.

In some embodiments, an origin of the 3D representation can be used to register the 3D representation with the medical device. For example, the point of origin of the 3D representation can be the patient's isocenter. This location can be defined during treatment planning and/or can be set by a user operating within a treatment planning software system and working with a 3D model of a patient (e.g., where the patient model is derived from a 3D dataset (e.g., CT or MRI scan), as described herein). In some embodiments, the origin of the 3D representation can be used to determine a treatment location (e.g., where radiation should be applied to the patient). For example, a set of operations, such as 3D shifts (e.g., (x, y, z) shifts), translations, etc., can be applied from the origin in order to align the patient such that the radiation is applied to a particular location on the patient (e.g., a location determined from the patient's treatment plan). In some embodiments, the set of operations can share the same coordinate system as the 3D representation, such that the operations can be specified in associated with the 3D representation.

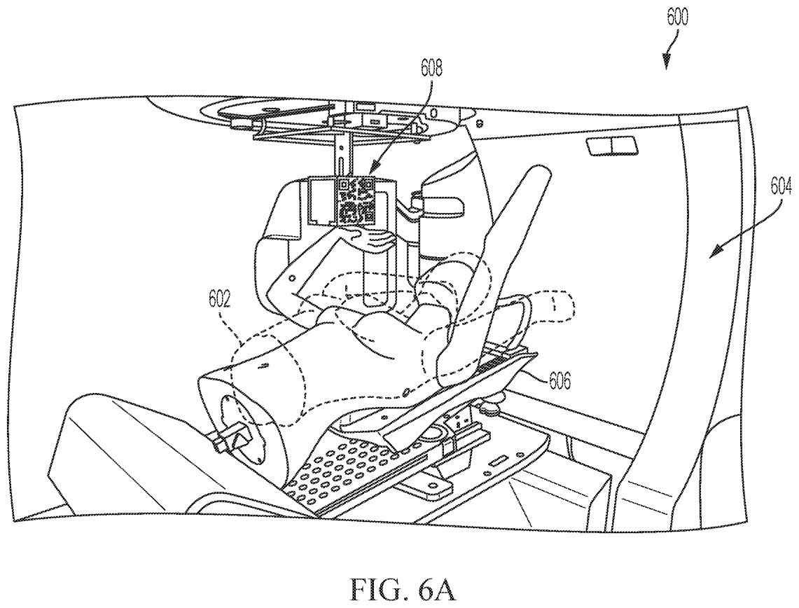

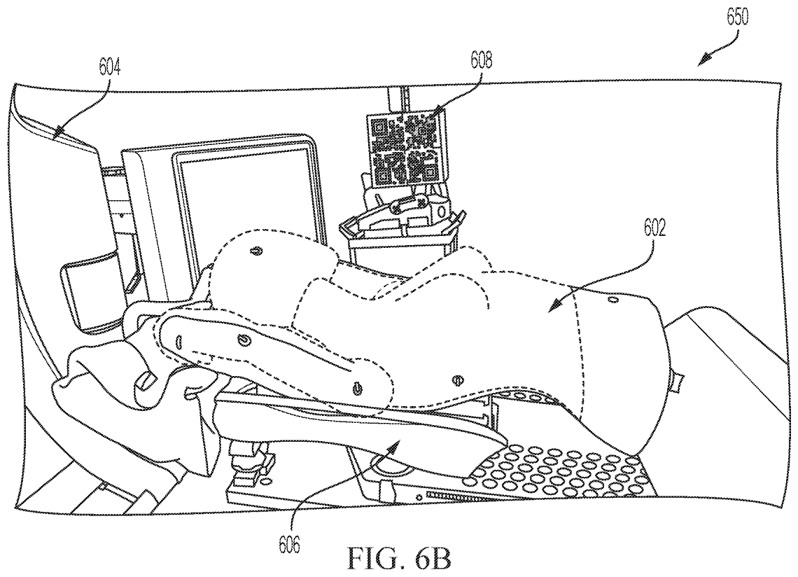

At step 412, the computing device generates, based on results of the registration process, a mixed reality visualization of the 3D representation of the patient and the medical device. In some embodiments, the computing device can render the 3D representation on the head mounted display to create a mixed reality visualization of the 3D representation in the physical scene. In some embodiments, the 3D representation can be rendered where the patient is to be positioned and aligned with the medical device. For illustrative purposes, FIGS. 6A-6B show a MixR rendering of a 3D representation of a person that is registered with the couch of a linear accelerator. FIG. 6A is an image 600 showing a 3D representation 602 of an anthropomorphic phantom from a first viewpoint in the physical scene where the phantom is mis-aligned according to the 3D representation, and FIG. 6B is an image 650 showing the 3D representation 602 of the anthropomorphic phantom from a second viewpoint in the physical scene whereby the phantom has been aligned to the 3D representation. As shown in images 600 and 650, the physical scene includes linear accelerator 604 with couch 606. The scene also includes tracking object 608, which in this example is mounted to the linear accelerator 604. The 3D representation 602 is registered with the linear accelerator 604 such that the 3D representation 602 is positioned on the couch in the pose to which the patient is to be aligned for treatment. While the example of FIGS. 6A-6B depicts the techniques being used with a linear accelerator, it should be appreciated that this is for exemplary purposes only, as the techniques are not so limited and can be used to align a patient with a myriad of medical devices.

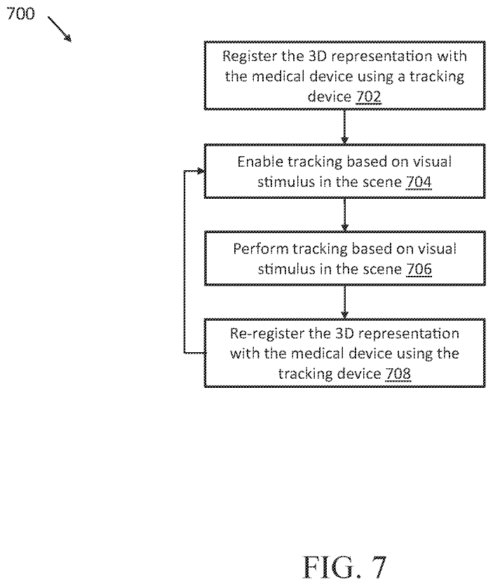

In some embodiments, the computing device performs the initial alignment of the 3D representation using marker-based tracking as described herein by leveraging the tracking object. In some embodiments, once the initial alignment is completed, the system can change tracking techniques to use marker-free tracking. FIG. 7 is a flow chart showing a computerized method 700 for registering the 3D representation and tracking the registration of the 3D representation over time, according to some embodiments. At step 702, the computing device performs the initial registration of the 3D representation using marker-based tracking (e.g., as described in conjunction with step 410). At step 704, the computing device transitions to instead perform marker-free tracking based on visual stimulus in the scene. For example, the computing device can enable visual simultaneous localization and mapping (VSLAM) tracking, which leverages the 3D structure of the physical environment to track the environment and maintain the registration of the 3D representation to the physical scene. Once VSLAM-based tracking is enabled, the system can track the environment without needing to use the tracking device.

In some embodiments, the computing device changes to using visual-based tracking based on whether the tracking object is captured in the images, based on inputs to the system (e.g., voice commands and/or other input commands), and/or the like. For example, when the tracking object is not in direct view of the head mounted display, and therefore not captured in the images acquired by the head mounted display, the system changes to marker-free tracking techniques. In some embodiments, voice commands can be used to transition from marker-based tracking to marker-free tracking techniques. The marker-free tracking techniques can be provided via the head mounted display (e.g., by passing a pose estimation to a native tracking algorithm used by the head mounted display) and/or by custom or third party tools.

At step 706, the computing device continues to track the scene using visual stimulus in the scene. The computing device obtains, via imaging devices on the head mounted display, images of the scene that include the visual stimulus, such as portion(s) of the medical device and/or other objects in the scene (and may still include the tracking device). The computing device tracks the pose of the 3D representation in the mixed reality visualization over time based on the visual stimulus in the images. The computing device uses the tracked pose over time to continuously update the mixed reality visualization so that the 3D representation can be perceived by the user at various poses in the environment as if the 3D representation were another physical object in the scene (e.g., as shown in FIGS. 6A-6B).

At step 708, the computing device transitions back to marker-based tracking and re-registers the 3D representation with the medical device using the tracking device. If the local environment changes, the 3D representation may drift as its location is being updated in relation to the surroundings, so changes in the surroundings can cause the 3D representation to move as well. If the 3D representation starts to drift, commands can be input to the computing device to cause the computing device to re-initialize the marker-based tracking techniques to re-anchor the 3D representation. As described herein, the computing device changes back to performing marker-based tracking by obtaining images of the scene with the tracking device and executing spatial tracking techniques to re-register the 3D representation with the medical device.

Once the 3D representation is re-registered with the medical device at step 708, the techniques can proceed back to step 704 and re-enable marker-free tracking techniques, as necessary. As described herein, the techniques can be used by clinicians to register a patient with a medical device in order to treat the patient. In some embodiments, the clinician can use the marker-based and/or marker-free techniques at different viewpoints in order to position the patient with the 3D representation. For example, the clinician can begin by viewing the tracking device from a first position in the room (e.g., from the left of the couch, right of the couch, superior of the couch, etc.). The clinician can register the 3D representation using marker-based tracking, and then transition to marker-free tracking in order to align the patient along one or more directions (e.g., left/right directions, anterior/posterior directions, superior/inferior directions, etc.). The clinician can then move to a new position in the room, re-register the 3D representation using the tracking object, transition to marker-free tracking, and further align the patient along one or more directions. Re-registering the 3D representation for each viewpoint can be used to ensure the 3D representation is accurately displayed in the MixR environment. For example, since VSLAM tracking uses the environment, when the patient is aligned at a particular step, it can change the overall environment and therefore also change the position of the 3D representation. Therefore, the re-registration process can be used to mitigate potential skew of the 3D representation that can be caused during the alignment process.

In some embodiments, the techniques can measure and/or quantify difference(s) between portion(s) of the 3D representation and the patient during patient alignment. Such differences can, for example, be used to provide guidance during patient alignment. The techniques can, in some embodiments, be used to determine distances from the user (e.g., the person performing patient alignment) to (1) the patient and (2) to the 3D representation in order to dynamically analyze and assess hologram-to-patient separation during patient alignment. Differences between the patient and the 3D representation can be visually indicated by one or more visual indicators. Such visual indicators can indicate portion(s) of the patient that are sufficiently aligned to the 3D representation, as well as portion(s) of the patient that require further adjustment in order to achieve proper alignment. The techniques can be performed in real-time during alignment, such that the user can see how subsequent patient adjustments improve (or worsen) the patient's alignment to the 3D representation.

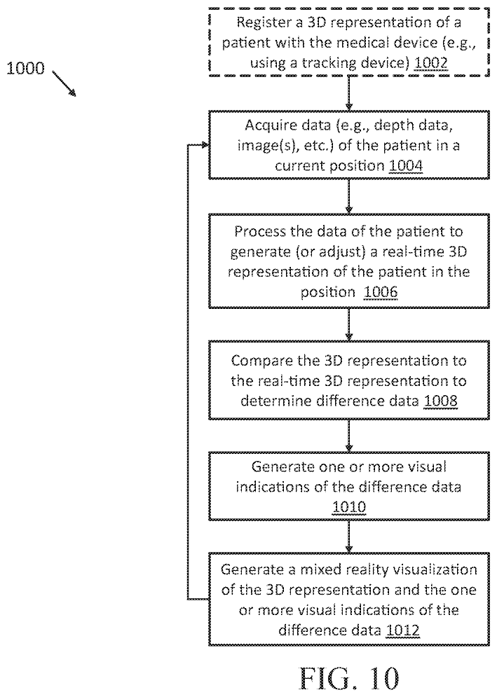

FIG. 10 is a flow chart of an exemplary computerized method 1000 for providing visual alignment indications to aid with aligning a patient with a 3D representation of the patient, according to some embodiments of the techniques described herein. At step 1002, the computing device performs a registration of the 3D representation to a medical device. In some embodiments, the registration can be performed using marker-based tracking (e.g., as described in conjunction with step 410). In some embodiments, the registration can be performed using visual stimulus in the scene (e.g., as described in conjunction with FIG. 7). Step 1002 is shown in dotted lines to indicate that step 1002 need not be performed each time. For example, if the 3D representation is already aligned with the medical device, then step 1002 can be omitted.

At step 1004, the computing device acquires data of the patient in a current position. For example, the computing device can capture the data while the patient is in a possible position for use with the medical device (e.g., while lying on a couch of a medical device). In some embodiments, the computing device can use depth sensor(s) to capture depth data associated with the patient. The depth sensor(s) can, for example, be disposed on a head-mounted display being worn by a user that is aligning the patient for treatment. Such depth sensor(s) can be time-of-flight range sensors and/or the like. Other types of data can additionally or alternatively be captured for use with the techniques described herein. For example, in some embodiments, the computing device can acquire real-time images of the patient in a current position with respect to the medical device. As a further example, the computing device can capture infrared (IR) data of the patient and/or use 3D imaging devices (e.g., which may or may not be associated with a HMD being worn by the user).

At step 1006, the computing device processes the data of the patient obtained at step 1004 to generate a real-time 3D representation of the patient in the current position. In some embodiments, the real-time 3D representation may be of a portion of the outer surface of the patient. In some embodiments, the real-time 3D representation represents distances of the patient's surface from a point of reference, such as from the user that is aligning the patient. Accordingly, the real-time 3D representation can effectively be a digital representation of the patient surface (e.g., which can be compared to the registered 3D representation, as described further herein). For example, a spatial mesh of the patient surface can be constructed using time-of-flight range data acquired at step 1004.

At step 1008, the computing device compares the registered 3D representation of the patient with the real-time 3D representation of the patient generated at step 1006 to determine difference data between the registered and real-time 3D representations. In some embodiments, the computing device may determine, for example, distances between one or more points or portions of the real-time 3D representation and associated points or portions of the registered 3D representation. For example, the computing device can compare known positions of points or locations of the registered 3D representation to positions of points or locations of the real-time 3D representation determined at step 1006. As a result, the computing device can measure the offset of points or locations between the registered and real-time 3D representations of the patient.

In some embodiments, the computing device can use, for example, ray-casting techniques to determine distance information. For example, ray casting can be used to determine distances between points of (1) the real-time 3D representation and (2) the registered 3D representation of the patient. Accordingly, ray casting can be used to assess the degree of alignment or overlap the 3D representations. In some embodiments, an origin point is determined for the rays (e.g., determined and/or accessed from computer storage). In some embodiments, for example, the origin of the rays can be the inside surface of a sphere centered on the radiation therapy target. Rays, such as evenly spaced rays, can be projected from the target. It should be appreciated that the rays are a conceptual concept, and therefore that actual rays need not be projected, rather the three-dimensional path represented by each ray can be traversed to perform the processing described herein. The computing device can determine the intersection points between a given ray and the registered 3D representation (e.g., the patient's hologram) and the real-time 3D representation (e.g., the patient's spatial mesh). The computing device can determine the difference between the points for each ray, to effectively determine the distance or difference between the 3D representations for each ray.

At step 1010, the computing device generates one or more visual indications of the difference data determined at step 1008. In some embodiments, the computing device can analyze or process the differences associated with the rays to classify or categorize each ray. Accordingly, the visual indication can indicate whether the intersection point of each ray (or a subset of the rays) with the real-time 3D representation is acceptable or not. While a number of different rays may be used, in some embodiments the computing device only processes a subset of the rays. For example, the computing device can ignore rays that are not near the perspective of the user (e.g., within a certain angle of the user's viewpoint).

In some embodiments, the computing device may categorize one or more portions of the real-time 3D representation (e.g., as represented by the determined ray distances or differences) based on one or more alignment thresholds and generate associated visual indications accordingly. The alignment thresholds can include, for example, a minimum distance indicative of an acceptable alignment, and one or more ranges indicative of varying levels of degrees of alignment (e.g., possibly unacceptable alignments). For example, two thresholds can be used that include (1) a first threshold indicative of an acceptable alignment within +/− a first distance and (2) a second threshold indicative of an alignment beyond the first distance. It should be appreciated that various other numbers of thresholds and/or threshold configurations can be used as well. For example, three thresholds can be used, which include (1) a first threshold indicative of an acceptable alignment (e.g., within +/− a first distance), (2) a second threshold indicative of an alignment too far within the registered 3D representation (e.g., greater than or equal to the negative (−) first distance) and (3) a third threshold indicative of an alignment too far beyond the registered 3D representation (e.g., greater than or equal to the positive (+) first distance). As another example, three thresholds can be used, which include (1) a first threshold indicative of an acceptable alignment (e.g., within +/− a first distance), (2) a second threshold indicative of a weak alignment (e.g., within +/− a second distance (e.g., the first distance plus an additional distance)), and (3) a third threshold indicative of a poor alignment (e.g., beyond +/− the second distance).

For each categorized portion of the real-time 3D representation, the computing device can generate an associated visual indication. For example, each threshold can be associated with a corresponding visual indication to visually indicate the different degrees of alignment. The visual indications can include different colors, patterns, lines, and/or the like. In some embodiments, the visual indications can be configured to indicate how the patient should be adjusted to achieve a better alignment. For example, for a portion of the patient that is too far within the registered 3D representation (e.g., where the ray intersects the real-time 3D representation before the registered 3D representation), a first visual indication can be used that comprises (e.g., that includes a first color and/or a first visual pattern). As another example, for a portion of the patient that is too far outside of the registered 3D representation (e.g., where the ray intersects the registered 3D representation before the real-time 3D representation), a second visual indication can be used (e.g., that includes a second color and/or a second visual pattern). For portions of the patient that are sufficiently aligned with the registered 3D representation (e.g., regardless of whether the ray(s) intersect the real-time or registered 3D representation first), a third visual indication can be used (e.g., that includes a third color and/or a third visual pattern).

In some embodiments, the visual indication can include a shape and/or color associated with each ray. For example, a checkered and/or polka dot pattern can be generated across the real-time 3D representation to convey difference data for each associated point of the 3D representation.

At step 1012, the computing device generates a mixed reality visualization of the 3D representation and the one or more visual indications of the difference data. In some embodiments as described herein, the computing device can render the 3D representation and the one or more visual indications on the head mounted display to create a mixed reality visualization of the 3D representation and the one or more visual indications in the physical scene. The visual indications can be displayed in conjunction with the registered 3D representation and/or the real-time 3D representation. For example, the visual indications can be displayed along a surface of the real-time 3D representation. As shown, step 1012 proceeds back to step 1004, such that the method described in conjunction with FIG. 10 can be performed iteratively during registration to provide for continued, real-time assessment of the surface registration accuracy with associated visual feedback as discussed herein. In some embodiments, for each subsequent iteration, aspects may be adjusted instead of generated from scratch. For example, at step 1006, the real-time 3D representation can be adjusted to reflect portion(s) of the user that moved, instead of generating the real-time 3D representation from scratch (e.g., since other portions of the patient may remain the same).

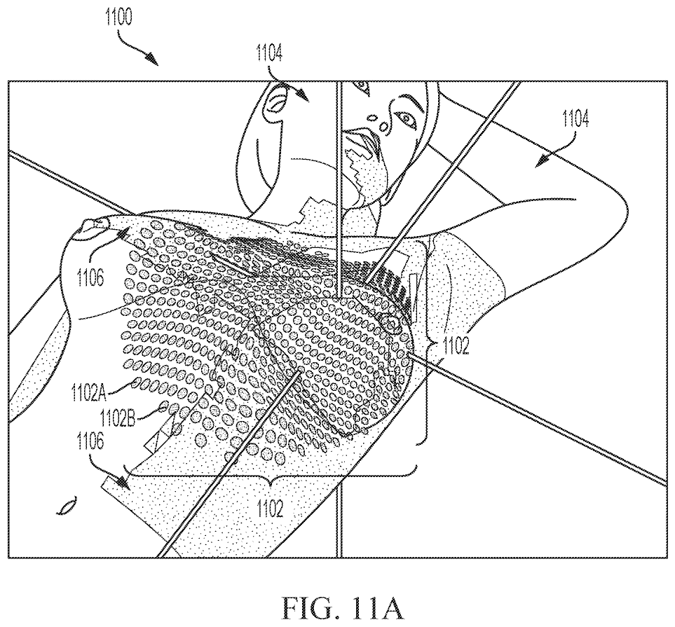

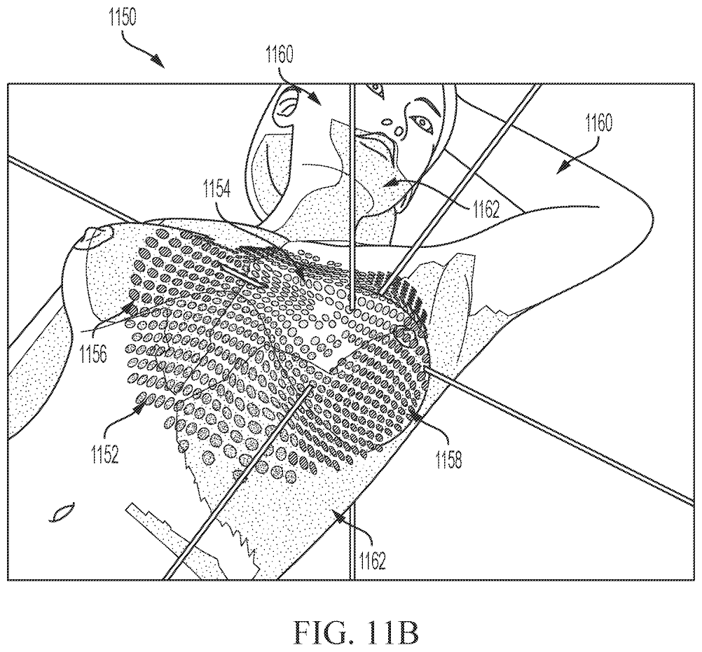

For illustrative purposes, FIGS. 11A-11B show a MixR rendering of a 3D representation of a person and a set of visual indicators. In particular, FIG. 11A is an image 1100 of a MixR rendering showing a visual indication 1102 of a portion of a patient that is aligned to a 3D representation of the patient within an acceptable alignment threshold, according to some embodiments of the techniques described herein. In this example, portions of the registered 3D representation 1104 are visible in the MixR rendering, along with portions of the real-time 3D representation 1106 of the patient at the current pose. In this example, the visual indication 1102 is indicative of a sufficient alignment of various points of the patient for purposes of treatment with the medical device. As described herein, the visual indication 1102 can include an associated pattern and/or color (e.g., green) that visually conveys the sufficient alignment in the MixR rendering. As described herein, ray casting can be used to assess associated points of the real-time and registered 3D representations. Accordingly, in this example, the visual indication 1102 includes a series of points (including points 1102A and 1102B), each of which is associated with a ray's intersection with the real-time 3D representation of the patient.

FIG. 11B is an image 1150 of a MixR rendering showing visual indications of both acceptable and unacceptable portions of the patient being aligned to the 3D representation, according to some embodiments of the techniques described herein. The image 1150 shows a visual indication 1152 of a portion of the real-time 3D representation that is sufficiently aligned with the registered 3D registration. The image 1150 also shows a visual indication 1154 of a portion of the real-time 3D representation that is too far outside of the registered 3D representation. The image 1150 also shows visual indications 1156 and 1158 of different portions of the real-time 3D representation that are too far within the registered 3D representation. As with FIG. 11A, portions of the registered 3D representation 1160 are visible in the MixR rendering, along with portions of the real-time 3D representation 1162 of the patient at the current pose.

In some embodiments, the techniques described herein can be used to display various types of information, including alone and/or in conjunction with the 3D representation. For example, various patient data can be rendered in the mixed reality visualization, such as images, medical data/information, and/or the like. As another example, to further aid alignment, the system can be configured to display photographs acquired during the initial setup at CT simulation. The displayed data can, for example, be linked to the hand movement of the user. Such hand movement-based techniques can enable a therapist to view the images and/or data as if held in their palm, which can provide quick back and forth viewing between the image and/or data and the patient. In some embodiments, the data can further be suspended in space, positioned, and oriented using hand gestures for viewing as desired by the user.

Various techniques are described herein for mixed-reality visualization (e.g., leveraging 3D representations, such as holograms) that can be used to align patients for medical applications. Such techniques can be used to visualize a patient and a 3D representation of the patient through, for example, an optical-see through or video-pass-through head mounted display. As described herein, such techniques can incorporate marker-based and/or marker-less tracking to initialize and orient the 3D representation of the patient as the user dynamically moves around the room during patient setup.

In some embodiments, the 3D representation can be registered to the patient by using sensors available on the head mounted display, which can be referred to as inside-out tracking. The inventors have discovered and appreciated that other tracking techniques can additionally or alternatively be incorporated that use hardware (beyond that of the head mounted display) to track and/or register objects. For example, external imaging device(s) and/or sensor(s) can be used to track a user, a patient, and/or objects in space, and to relay navigational cues to the user via a display system. Use of such external hardware can be referred to as outside-in tracking. As an illustrative example, SGRT utilizes ceiling-mounted light emitters and optical cameras to project a known light pattern onto the patient's skin that is reflected and captured by the optical cameras. Thereafter, the patient surface can be reconstructed (e.g., in 3D) and displayed on a conventional 2D monitor in comparison to a reference surface.

The inventors have appreciated that it can be desirable to additionally or alternatively use other hardware beyond that of the head mounted display to perform and/or aid in tracking and spatial mapping of the patient. In particular, the inventors have appreciated that outside-in spatial mapping and/or tracking can be used to achieve high resolution patient surface reconstructions, which can be used to provide quantitative comparisons by analyzing differences between the reconstructed patient surface and a reference surface.

In some embodiments, the techniques described herein can be implemented using, at least in part, outside-in tracking. For example, SGRT systems can be augmented to integrate mixed-reality visualization as described herein that use outside-in tracking approaches. In some embodiments, outside-in tracking can additionally or alternatively be used to visualize patient surfaces. For example, 3D patient surfaces can be extracted from external cameras, such as those of the SORT systems. Such patient surfaces can be displayed through a head-mounted display. In some embodiments, markers (e.g., infrared and/or electromagnetic markers) can be attached to the head-mounted display, and then tracked in space using fixed sensors. As a result, the external sensors can locate the user in its frame of reference, which can be calibrated to share the same frame of reference as the therapy delivery system.

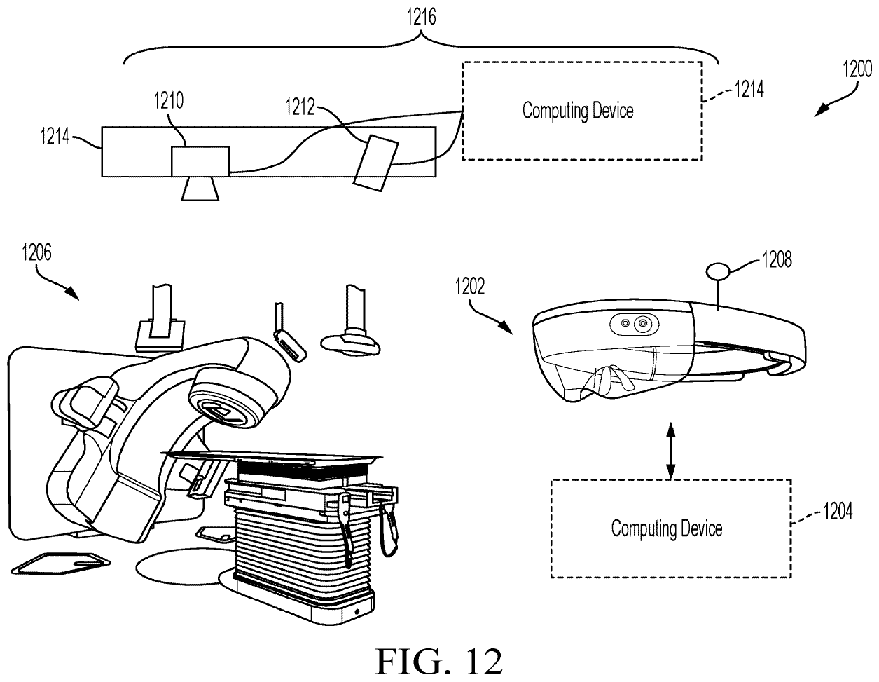

FIG. 12 is a diagram of an exemplary system 1200 for registering a 3D representation of a patient with a medical device, according to some embodiments. The system 1200 includes a head mounted display 1202 with a computing device 1204 (e.g., which can be the head mounted display 102 and/or computing device 104 of FIG. 1). The system 1200 also includes a medical device 1206 to which the patient is to be aligned, which is shown as a linear accelerator, but the techniques are not so limited as described herein. The head mounted display 1202 also includes a marker 1208 that is mounted to the head mounted display 1202. While only one marker 1208 is shown in FIG. 12, it should be appreciated that any number of marker(s) can be mounted to the head mounted display 1202. The marker 1208 can be, for example, an image-based optical tracker, a shape-based optical tracker, a radio frequency tracker, an infrared-based tracker, and/or the like.

The system 1216 includes an external tracking system 1216, which includes imaging device 1210 (e.g., optical camera(s)), illumination device 1212 (e.g., light emitter(s), projector(s), etc.), and computing device 1214 in communication with imaging device 1210 and illumination device 1212. As shown in FIG. 12, the imaging device 1210 and illumination device 1212 can be mounted on a standing rack 1214 in the room in which the medical device 1206 is located. The example shown in FIG. 12 includes just a single imaging device 1210 and illumination device 1212, although it should be appreciated that any number of imaging device(s) or illumination device(s) can be used in accordance with the techniques described herein. It should also be appreciated that the imaging device(s) and/or illumination device(s) can be mounted in locations other than the rack 1214, such as on the ceiling of the room, on the wall(s) of the room, etc. It should be further appreciated that while separate computing devices 1204 and 1214 are shown in FIG. 12, just a single computing device may be used and/or additional computing devices, whether local and/or remote, can likewise be used with the techniques described herein.

In some embodiments, the system 1200 can use the medical device 1206 and/or the external tracking system 1216 to monitor the position of the patient and/or track the pose of the head mounted display 1202 by tracking the marker 1208. For example, the tracking system 1216 can measure and/or monitor the surface of the patient. As another example, the external tracking system 1216 can measure and/or track the pose of the head mounted display 1202. However, this is for exemplary purposes and various configurations can be used to achieve the techniques described herein.

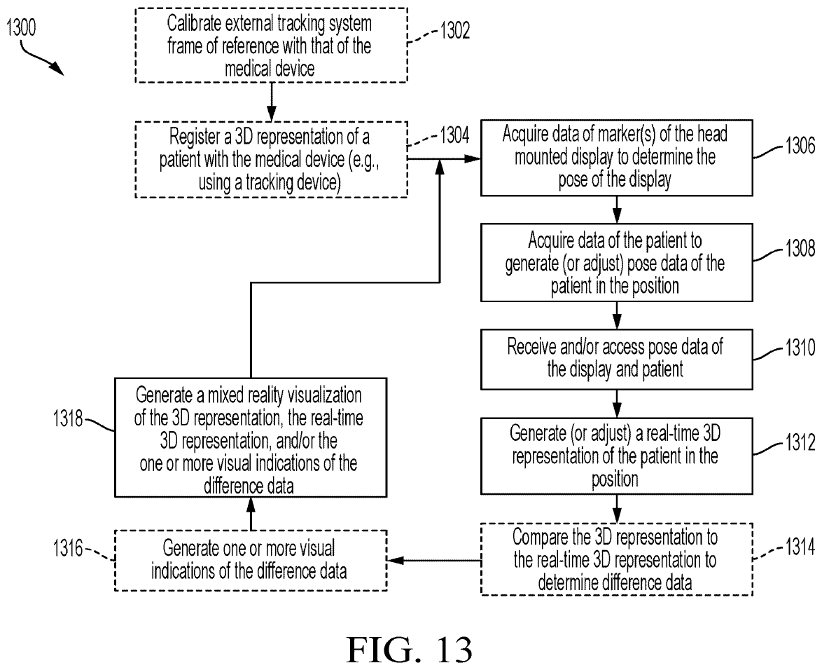

FIG. 13 is a flow chart of an exemplary computerized method 1300 for generating a mixed reality visualization of a 3D representation of the patient and the medical device using outside-in tracking, according to some embodiments. Referring to FIGS. 12-13, at step 1302, the frame of reference of the external tracking system 1216 is calibrated with the frame of reference of the medical device 1206. In some embodiments, for example, the center of a physical phantom can be aligned with the isocenter of the medical device 1206 using the therapy system's on-board imaging. In some examples, the physical phantom can have tracking sensors attached with a known offset to the center of the physical phantom, thus establishing the relationship between the external tracking system 1216 and the isocenter of the medical device 1206. Step 1302 is shown in dotted lines to indicate that step 1302 need not be performed each time. For example, if frames of reference are already calibrated, then step 1302 can be omitted.

At step 1304, the system 1200 performs a registration of a 3D representation of the patient to the medical device 1206. In some embodiments, the registration can be performed using marker-based tracking (e.g., as described in conjunction with step 410). In some embodiments, the registration can be performed using visual stimulus in the scene (e.g., as described in conjunction with FIG. 7). Step 1304 is shown in dotted lines to indicate that step 1304 need not be performed each time. For example, if a 3D representation is already aligned with the medical device, then step 1304 can be omitted. In some embodiments, the registration can be performed using the external tracking system 1216 and/or by using the head mounted display 1202 and computing device 1204.

At step 1306, the external tracking system 1216 acquires data of the marker 1208 and determines pose information of the head mounted display 1202. In some embodiments, the pose information of the head mounted display 1202 is indicative of a relative spatial relationship between the marker 1208 associated with the head mounted display 1202 and the medical device 1206. The relative spatial relationship between the marker 1208 and the medical device 1206 can indicate a relative pose of (a) the head mounted display 1202 to the medical device, (b) the medical device to the head mounted display 1202, or some combination thereof. The relative spatial relationship can include a transformation, a mapping, and/or the like.

At step 1308, the system 1200 (e.g., the external tracking system 1216) acquires data of the patient in a current position. For example, the external tracking system 1216 can illuminate the patient and can capture images of the surface of the patient while the patient is in a possible position for use with the medical device (e.g., while lying on a couch of a medical device). In some embodiments, the external tracking system 1216 can additionally or alternatively use depth sensor(s) to capture depth data associated with the patient as described herein.

At step 1310, the head mounted display receives and/or accesses the pose data of the head mounted display 1202 at step 1306 and pose data of the position of the patient determined at step 1308. At step 1312, the head mounted display 1202 (e.g., using computing device 1204) can process the pose data of the patient to generate or adjust a real-time 3D representation of the patient in the current position. In some embodiments, the real-time 3D representation may be of a portion of the outer surface of the patient as described herein. In some embodiments, the head mounted display 1202 can use the pose data of the head mounted display from step 1306 and the pose data of the position of the patient from step 1308, at least in part, to update over time the pose of the real-time 3D representation. Additionally or alternatively, the head mounted display 1202 can use the pose data of the head mounted display from step 1306 and the pose data of the position of the patient from step 1308, at least in part, to update over time the pose of the 3D representation of the patient.

As described herein, optionally the head mounted display 1202 can provide visual alignment indications to aid with aligning the patient (using the real-time 3D representation) with the 3D representation of the patient. At step 1314, the computing device 1204 optionally compares the registered 3D representation of the patient with the real-time 3D representation of the patient generated at step 1312 to determine difference data between the registered and real-time 3D representations. In some embodiments, as described herein, the computing device 1204 can measure the offset of points or locations between the registered and real-time 3D representations of the patient. At step 1316, the computing device 1204 can optionally generate one or more visual indications of the difference data determined at step 1314, as also described herein (e.g., shapes, patterns, colors, etc.).

At step 1318, the computing device generates a mixed reality visualization of the registered 3D representation, the real-time 3D representation, and/or the one or more visual indications of the difference data. In some embodiments as described herein, the computing device can render the 3D representation and the one or more visual indications on the head mounted display to create a mixed reality visualization of the 3D representation and the one or more visual indications in the physical scene. The visual indications can be displayed in conjunction with the registered 3D representation and/or the real-time 3D representation, as described herein. As shown, step 1318 proceeds back to step 1306, such that the method described in conjunction with FIG. 13 can be performed iteratively to provide for continued, real-time assessment of the surface registration accuracy (optionally, with associated visual feedback) as discussed herein. In some embodiments, for each subsequent iteration, aspects may be adjusted instead of generated from scratch. For example, at step 1312, the real-time 3D representation can be adjusted to reflect portion(s) of the user that moved and/or to maintain consistent tracking accuracy over time, instead of generating the real-time 3D representation from scratch.

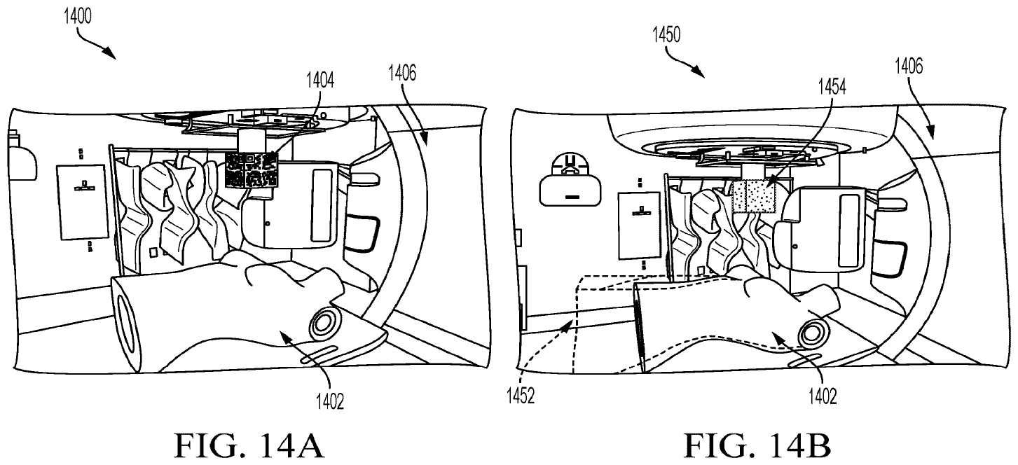

FIGS. 14A and 14B show exemplary images of a real-world scene 1400 and a MixR rendering 1450 to illustrate the use of a 3D representation of a marker 1404 for alignment verification, according to some embodiments. FIG. 14A shows a real-world image that includes an anthropomorphic phantom 1402 aligned with a medical device 1406. The real-world scene 1400 also shows a marker 1404, similar to the marker 608 shown in FIGS. 6A and 6B. 3D representations 1452, 1454 of both the anthropomorphic phantom 1402 and the marker 1404 may be rendered and registered with the medical device 1406. Registration refers to a marker-based process, similar to the process discussed with reference to step 410, for example. Specifically, alignment between the marker 1404 and its 3D representation 1454 is ensured and calibration is performed as needed.

FIG. 14B shows a MixR rendering 1450 that includes a 3D representation 1452 of the anthropomorphic phantom 1402, as well as a 3D representation 1454 of the marker 1404 that are registered with the medical device 1406. Following registration, marker-free tracking may be performed to update the MixR rendering 1450. Because the MixR rendering 1450 includes both the marker 1404 and its 3D representation 1454, if the alignment of the 3D representation 1454 begins to drift (e.g., when using marker-free tracking) or an issue develops in a camera of a head-mounted display used to view the MixR rendering 1450(e.g., and thus there is a deviation when using marker-based tracking), for example, then the 3D representation 1454 will visibly deviate from the marker 1404. Thus, the misalignment will be visually apparent to a user wearing a head-mounted display. When the user perceives a misalignment when using marker-free tracking, the user may issue a command (e.g., a voice command) to re-register the 3D representations 1452, 1454 as discussed with reference to step 704 of FIG. 7. Accordingly, misalignment that occurs during marker-free tracking can be fixed by performing a re-registration process using marker-based tracking.

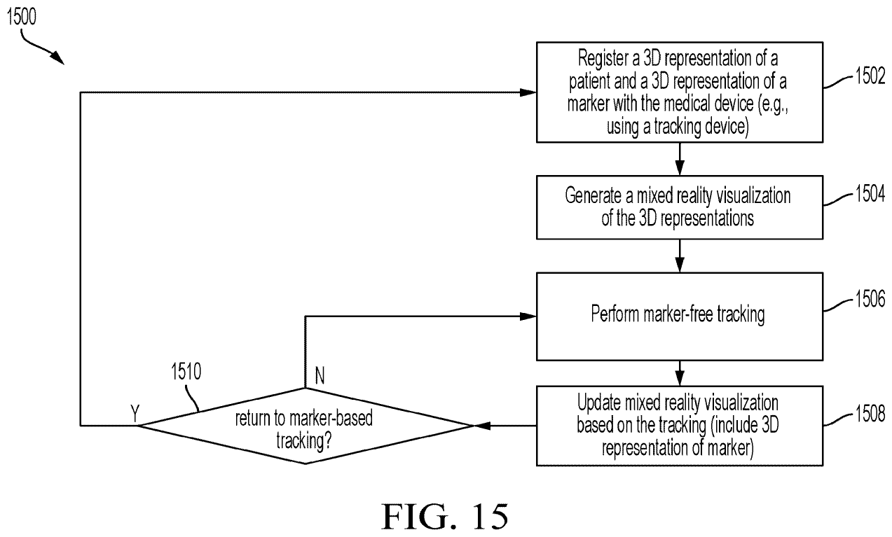

FIG. 15 is a flow chart of an exemplary computerized method 1500 for confirming registration of 3D representations with the medical device during mixR rendering, according to some embodiments of the techniques described herein. The method 1500 combines aspects of the methods 400, 700, 1000 discussed with reference to FIGS. 4, 7, and 10. The method may be combined with or may modify the method 1300 of FIG. 13, for example, to ensure that the mixR visualization maintains alignment between 3D representations and the real-world coordinate system during outside-in tracking to position a patient.

At step 1502, the computing device performs a registration of a 3D representation of a patient and also of a 3D representation of a marker (e.g., tracking device 1404) to a medical device. This registration may be similar to the determination of relative pose information discussed with reference to step 408 but additionally involves having a 3D representation of the tracking device or marker. As previously noted, the marker may be an image-based optical tracker, shape-based optical tracker, radio frequency or IR tracker, and/or the like. The marker has a fixed position such that its position relative to the medical device is fixed.

At step 1504, the computing device generates a MixR visualization of the 3D representations registered at step 1502. At step 1506, performing marker-free tracking is similar to the data acquisition discussed for step 1004 in FIG. 10 or for step 1308 of FIG. 13. Marker-free tracking may refer to tracking based on visual stimulus in the scene, as discussed for step 704, for example. Based on the marker-free tracking, the computing device updates the MixR visualization, at step 1508, that shows the 3D representation of the patient and also shows the 3D representation of the marker.

This additional inclusion of the 3D representation of the marker allows the user wearing the head-mounted display to determine whether a misalignment has developed since the latest registration (at step 1502). That is, the user can readily see the alignment status between the MixR visualization and real-world coordinate system. If misalignment is apparent, the user may then issue a command (e.g., a voice command) to return to marker-based tracking (i.e., re-registration).

At step 1510, the computing device may check whether a command is issued to return to marker-based tracking based on the user of the head-mounted display determining that the 3D representation of the marker is misaligned with the marker in the latest update of the MixR visualization (at step 1508). If the check indicates the command, then re-registration of the 3D representations of the patient and the marker is performed at step 1502 to start the next iteration. If the check indicates the absence of a command (i.e., no misalignment), then marker-free tracking continues at step 1506 for the next iteration.

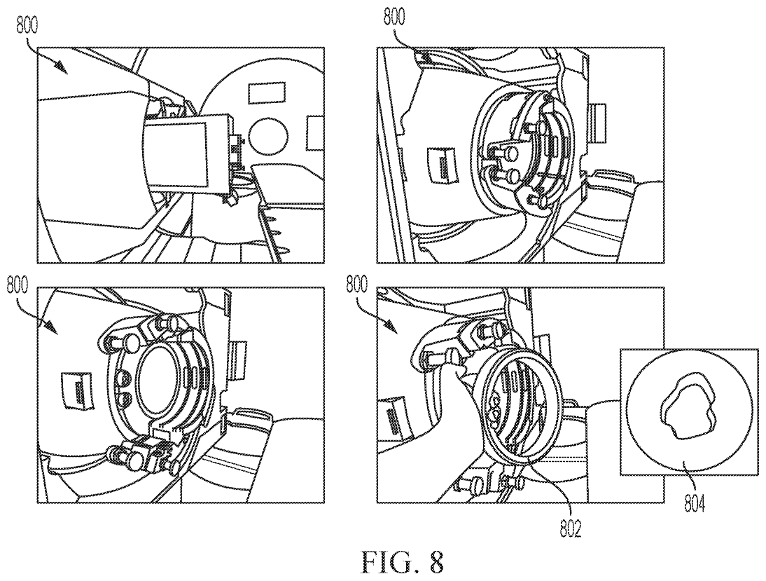

While some examples provided herein are described in the context of linear accelerators, techniques can be used with other types of medical devices. FIG. 8 includes images of an exemplary proton therapy device 800 that can be used with the 3D representation registration techniques described herein, according to some embodiments. The tracking device can be mounted to the proton therapy device 800 as described herein, including leveraging existing components of the proton therapy device (e.g., the rings 802 and 804) and/or additional components that can be used to mount the tracking device to the proton therapy device 800. In this example, the rings 802 and/or 804 are brass apertures, which can be milled to include necessary holes and/or components to mount the tracking device to the brass aperture.

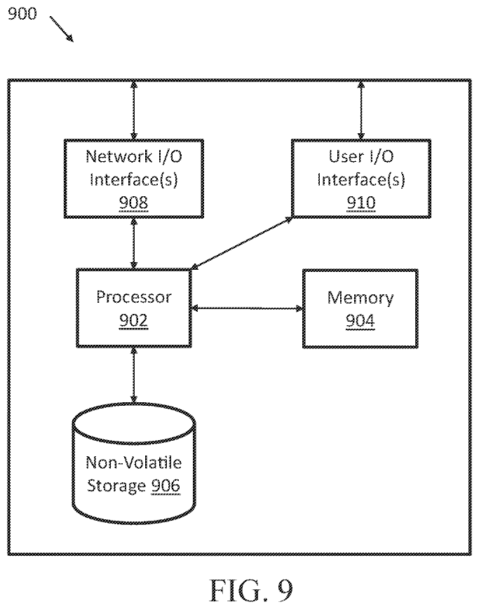

An illustrative implementation of a computer system 900 that may be used in connection with any of the embodiments of the disclosure provided herein is shown in FIG. 9. For example, the computer system 900 can be used for the computing device 102 in FIG. 1. The computer system 900 may include one or more computer hardware processors 902 and one or more articles of manufacture that comprise non-transitory computer-readable storage media (e.g., memory 904 and one or more non-volatile storage devices 906). The processor 902 (s) may control writing data to and reading data from the memory 904 and the non-volatile storage device(s) 906 in any suitable manner. To perform any of the functionality described herein, the processor(s) 902 may execute one or more processor-executable instructions stored in one or more non-transitory computer-readable storage media (e.g., the memory 904), which may serve as non-transitory computer-readable storage media storing processor-executable instructions for execution by the processor(s) 902. The computer system 900 may include various input/output (I/O) interfaces to interface with external systems and/or devices, including network I/O interface(s) 908 and user I/) interface(s) 910.

The computer system 900 can be any type of computing device with a processor 902, memory 904, and non-volatile storage device 906. For example, the computer system 900 can be a server, desktop computer, a laptop, a tablet, or a smartphone. In some embodiments, the computer system 900 can be implemented using a plurality of computing devices, such as a cluster of computing devices, virtual computing devices, and/or cloud computing devices.

The terms “program” or “software” are used herein in a generic sense to refer to any type of computer code or set of processor-executable instructions that can be employed to program a computer or other processor (physical or virtual) to implement various aspects of embodiments as discussed above. Additionally, according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, for example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term). The phraseology and terminology used herein is for the purpose of description and should not be regarded as limiting. The use of “including,” “comprising,” “having.” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A method of registering a patient with a medical device for treating the patient, the method comprising:
accessing pose information, wherein the pose information is of a patient, a mixed reality display, or both, and wherein the pose information is generated by a tracking system external to the mixed reality display;
registering a pose of a 3D representation of the patient with the medical device using, at least in part, the pose information; and
generating, based on results of the registering, a mixed reality visualization of the 3D representation of the patient and the medical device.

2. The method of claim 1, wherein:
the pose information of the mixed reality display is indicative of a relative spatial relationship between a marker associated with the mixed reality display and the medical device; and
the relative spatial relationship between the marker and the medical device indicates a relative pose of (a) the head mounted display to the medical device, (b) the medical device to the head mounted display, or some combination thereof.

3. The method of claim 2, wherein the relative spatial relationship comprises a transformation, a mapping, or some combination thereof.

4. The method of claim 2, wherein the relative pose information is determined based on the marker being mounted to the mixed reality display.

5. The method of claim 2, wherein the marker comprises an image-based optical tracker, a shape-based optical tracker, a radio frequency tracker, an infrared-based tracker, or some combination thereof.

6. The method of claim 2, further comprising:
registering a 3D representation of the marker with the medical device, wherein the marker is in a fixed relationship with the medical device.

7. The method of claim 6, wherein the mixed reality visualization includes the 3D representation of the marker.

8. The method of claim 7, further comprising:
performing tracking on visual stimulus in the scene; and
updating the mixed reality visualization including the 3D representation of the patient and the 3D representation of the marker, wherein the mixed reality visualization allows a user to see an alignment status of the 3D representation of the marker with the marker.

9. The method of claim 8, further comprising:
re-registering the 3D representation of the patient and the 3D representation of the marker based on the relative spatial relationship between the marker and the medical device based on a request from the user.

10. The method of claim 1, further comprising perform tracking of the 3D representation, comprising:
accessing second pose information, wherein:
the second pose information is of the patient, the mixed reality display, or both, at a second time occurring after a first time associated with the pose information; and
the pose information is generated by a tracking system external to the mixed reality display;
tracking the pose of the 3D representation in the mixed reality visualization over time based on the second pose information.

11. The method of claim 1, further comprising:
generating the mixed reality visualization of the 3D representation of the patient, the patient, and a visual indication indicative of an alignment of the patient with the 3D representation.

12. The method of claim 11, wherein the visual indication is indicative of:
a portion of the patient that is aligned to the 3D representation within a threshold; or
the portion of the patient that is not aligned to the 3D representation within the threshold.

13. The method of claim 11, further comprising generating the visual indication, comprising:
accessing data of the patient in a current position from the external tracking system;
processing the data to generate a real-time 3D representation of at least a portion of the patient in the current position;
compare the real-time 3D representation to the 3D representation to determine difference data; and
generate the visual indication based on the difference data.

14. The method of claim 1, wherein the instructions are further configured to cause the at least one computer hardware processor to render a 3D representation of the generated mixed reality visualization.

15. The method of claim 1, wherein registering the 3D representation of the patient with the medical device comprises mapping a pose of the 3D representation to a machine coordinate space.

16. The method of claim 1, wherein:
the medical device is a radiotherapy device, wherein the radiotherapy device comprises a couch on which the patient lies; and
generating the mixed reality visualization comprises generating the mixed reality visualization with the 3D representation at a pose such that the 3D representation is on the couch of the radiotherapy device.

17. At least one computer readable storage medium storing processor-executable instructions that, when executed by at least one processor, cause the at least one processor to perform:
accessing pose information, wherein the pose information is of a patient, a mixed reality display, or both, and wherein the pose information is generated by a tracking system external to the mixed reality display;
registering a pose of a 3D representation of the patient with a medical device using, at least in part, the pose information; and
generating, based on results of the registering, a mixed reality visualization of the 3D representation of the patient and the medical device.

18. An apparatus for registering a 3D representation of a patient with a medical device, the apparatus comprising:
at least one computer hardware processor; and
at least one non-transitory computer-readable storage medium storing processor executable instructions that, when executed by the at least one computer hardware processor, cause the at least one computer hardware processor to perform:
accessing pose information, wherein the pose information is of a patient, a mixed reality display, or both, and wherein the pose information is generated by a tracking system external to the mixed reality display;
registering a pose of a 3D representation of the patient with a medical device using, at least in part, the pose information; and
generating, based on results of the registering, a mixed reality visualization of the 3D representation of the patient and the medical device.
